(19) United States
(12) Patent Application Publication
(10) Pub. No.: US 2014/0316012 A1
(43) Pub. Date: Oct. 23, 2014

(54) In-Situ Forming Foams for Embolizing or Occluding a Cavity

(71) Applicants: Toby Freyman, Lexington, MA (US); Jennifer Mortensen, Somerville, MA (US)

(72) Inventors: Toby Freyman, Lexington, MA (US); Jennifer Mortensen, Somerville, MA (US)

(21) Appl. No.: 14/205,768

(22) Filed: Mar. 12, 2014

Related U.S. Application Data
(60) Provisional application No. 61/852,432, filed on Mar. 15, 2013.
(60) Provisional application No. 61/852,339, filed on Mar. 15, 2013.

(51) Int. Cl. A61L 31/14

(52) U.S. Cl. CPC A61L 31/146; A61L 2400/06
USPC 514/772.3

(57) ABSTRACT

The present invention provides systems and methods for occluding and/or embolizing a cavity within a patient by delivering a prepolymer material into or onto a cavity and forming an expanding foam within the cavity. The inventions methods are applicable to occluding a variety of cavities, including blood vessels, aneurysms, left arterial appendages, vascular malformations and the like.

### CROSS-REFERENCE TO RELATED APPLICATIONS

This application claims priority to U.S. Provisional Patent Application Ser. No. 61/852,432 filed Mar. 15, 2013, entitled “In-situ Forming Foams For Treatment Of The Left Arterial Appendage.” This application also claims priority to U.S. Provisional Patent Application Ser. No. 61/852,339 filed Mar. 15, 2013, titled “Commercial Applications of In-situ Forming Foam Implants.” Each of the foregoing applications is incorporated herein by reference for all purposes.

### FIELD OF THE INVENTION

Systems and methods related to the use of in-situ forming foams for embolization are generally described. The foams can be applied to the interior of a blood vessel or other cavity for purposes of embolization or generally occluding or filling a tubular structure or other cavity in the body. Upon deployment in the vessel or cavity, the forming foam provides an efficacious means of embolization.

### BACKGROUND

Embolization of blood vessels or other lumens or cavities is a common and necessary treatment and has a number of clinical applications, including tumor reduction and treating vascular malformations and aneurysms. For example, embolization may be necessary for treatment in connection with: i) bleeding after a dilation and curettage (D&C) procedure, ii) post-hysterectomy bleeding, iii) uterine AV fistulas, iv) liver or lung resection; v) HHT fistula; vi) gastrointestinal bleeding; vii) pre-, intra- or post-operative hemorrhages; viii) arteriovenous malformations; ix) endovascular repair of aneurysms; and x) uterine artery embolization. However, current means of embolization may have limitations such as the extent to which they fill the vessel, control drug delivery, and conform in shape to complex anatomies. What is are methods and compositions that more completely fill blood vessels or other bodily lumens or cavities in need of embolization.

### SUMMARY OF THE INVENTION

For the purposes of this disclosure, the terms “formulation”, “prepolymer” and “prepolymer formulation” are used interchangeably to designate a polymer-based system or material capable of further reaction in a vessel or cavity. As used herein, “cavity” is used interchangeably with “lumen” to mean a space within the body that may be occluded or embolized. These terms can refer to a single prepolymer material, or a prepolymer material blended with other additives (e.g., catalysts, surfactants, solvents, diluents, crosslinkers, chain extenders, blowing agents) to create a prepolymer formulation. The polymers and foams that are used in the embodiments of the present invention may be any of those disclosed in U.S. application Ser. No. 13/209,020, filed Aug. 12, 2011 and titled “In-situ Forming Hemostatic Foam Implants,” which is a continuation-in-part of U.S. Application Ser. No. 12/862,362, filed Aug. 24, 2010 and titled “Systems and Methods Relating to Polymer Foams,” which claims priority to U.S. Provisional Patent Application Ser. No. 61/236,314 filed Aug. 24, 2009, titled “Systems and Methods Relating to Polymer Foams,” each of which are incorporated by reference herein for all purposes. Also incorporated by reference is the commonly-assigned U.S. patent application entitled “In-situ Forming Foams with Outer Layer,” filed concurrently herewith and naming Freyman et al. as in inventors.

In one aspect, the present invention relates to methods and systems for occluding a cavity within a patient comprising: providing a fluid prepolymer material, delivering the fluid prepolymer material into (or onto) a cavity and forming a foam within the cavity from the fluid prepolymer material. As used herein, “cavity” is used interchangeably with “lumen” to mean a space within the body that may be occluded or embolized. In one embodiment, the cavity is a blood vessel, vascular malformation or left arterial appendage. In one embodiment, the foam embolizes the cavity to prevent or stop bleeding. In one embodiment, the fluid prepolymer material is delivered using a catheter, endoscope or related minimally-invasive medical device. In one embodiment, the foam is an expanding foam. As used herein, the term “patient” or “subject” refers to both human and non-human organisms. The foams of the present invention are described as being formed “in-situ” because they are formed after the delivery of one or more prepolymers to the site of the cavity, as further described herein.

In one aspect, the present invention comprises a system comprising an insertable medical device and a one-, two- or multi-part in-situ forming foam. The medical device comprises a structure having a first end, a second end, and an exterior surface between the first and second ends. The in-situ forming foam comprises a formulation that reacts in-situ (i) between formula constituents, and/or (ii) in the presence of an aqueous environment (e.g., blood, water, etc.), and/or (iii) as triggered by biological environmental factors such as temperature, pH, salinity, osmotic pressure, and the like, to generate a gas and form the foam. When used in the system as an embolic in a vessel or cavity, the foam is in contact with at least a portion of the exterior surface of the medical device and/or the interior surface of the vessel or cavity. When used in the system to treat a left atrial appendage (“LAA”), the foam is in contact with at least a portion of the exterior surface of the medical device and/or the tissue surface of the LAA. The foam reacts and solidifies to, among other things, prevent and treat blood clots.

In another aspect, the present invention comprises a method comprising the use of in-situ forming foam as an embolic in a vessel or cavity. The foam reacts, preferably forms a coil or other suitable form, and expands to, among other things, embolize a vessel, tubular lumen or cavity.

In another aspect, the present invention comprises a kit that includes a medical device and a formulation. The medical device comprises a structure having a first end, a second end, and an exterior surface between the first and second ends. The formulation reacts by the combination of formulation constituents, and/or by exposure to an aqueous-containing environment (e.g., blood or water), in either case to generate a gas and form a foam. According to certain embodiments, such kits may also contain one or more traditional embolization devices (e.g., coils, spheres, etc.) for use in conjunction with the foams.

In another aspect, the present invention comprises instructions for embolizing a vessel or cavity. The instructions instruct a healthcare provider to insert one or more prepolymer materials within the vessel or cavity, where the prepolymer materials react by the combination of formulation constituents, and/or by exposure to an aqueous-containing environment (e.g., blood or water), in either case to generate a gas and form a foam.

In another aspect, the present invention comprises instructions for treating a LAA. The instructions instruct a healthcare provider to place a medical device within the LAA and to insert an in situ forming foam within the LAA, where the in situ forming foam comprises a formulation that reacts in the presence of an aqueous environment to generate a gas and form a foam.

In another aspect, the invention includes foams, compositions, formulations, products, kits, and systems that are useful for providing the foams and performing the methods described above.

### BRIEF DESCRIPTION OF THE DRAWINGS

The above and further advantages of the invention may be better understood by referring to the following description in conjunction with the accompanying drawings, in which:

FIG. 1 depicts a foam coil that has expanded to fill an aneurysm, in accordance with an embodiment of the present invention.

FIG. 2 depicts a polymer formulation delivered from a catheter such that it forms a foam coil that expands to diameter or length greater than the inner lumen of the catheter, in accordance with an embodiment of the present invention.

FIG. 3 depicts a delivery catheter with a design feature at its distal end that provides a weakened area along the coil to facilitate detachment of the coil, in accordance with an embodiment of the present invention.

FIG. 4 depicts a delivery catheter in which a balloon is incorporated within the lumen of the catheter such that the diameter of the polymer solution is reduced or entirely blocked, thereby establishing a break between the deployed polymer solution and the polymer solution remaining in the lumen of the catheter, in accordance with an embodiment of the present invention.

FIG. 5 depicts a delivery catheter comprising a non-circumferential hydrophilic or moisture permeable material such that preferential surface curing occurs on only a portion of the circumference of the polymer surface, thereby leading to a coiling of the foam coil upon delivery from the catheter, in accordance with an embodiment of the present invention.

FIG. 6 depicts the cross-section of a coil resulting from a delivery catheter comprising four discrete hydrophilic or moisture-permeable regions spaced evenly (i.e., equidistant) around the circumference of the catheter lumen, in accordance with an embodiment of the present invention.

FIG. 7 depicts a catheter tip comprising a balloon or hood that constrains expansion of the foam to the area within a left arterial appendage (LAA) during delivery of the polymer solution and/or foam formation, in accordance with an embodiment of the present invention.

FIG. 8 depicts an arteriovenous malformation that may be treated by embolization, in accordance with an embodiment of the present invention.

### DETAILED DESCRIPTION OF THE PREFERRED EMBODIMENTS

### Embolization

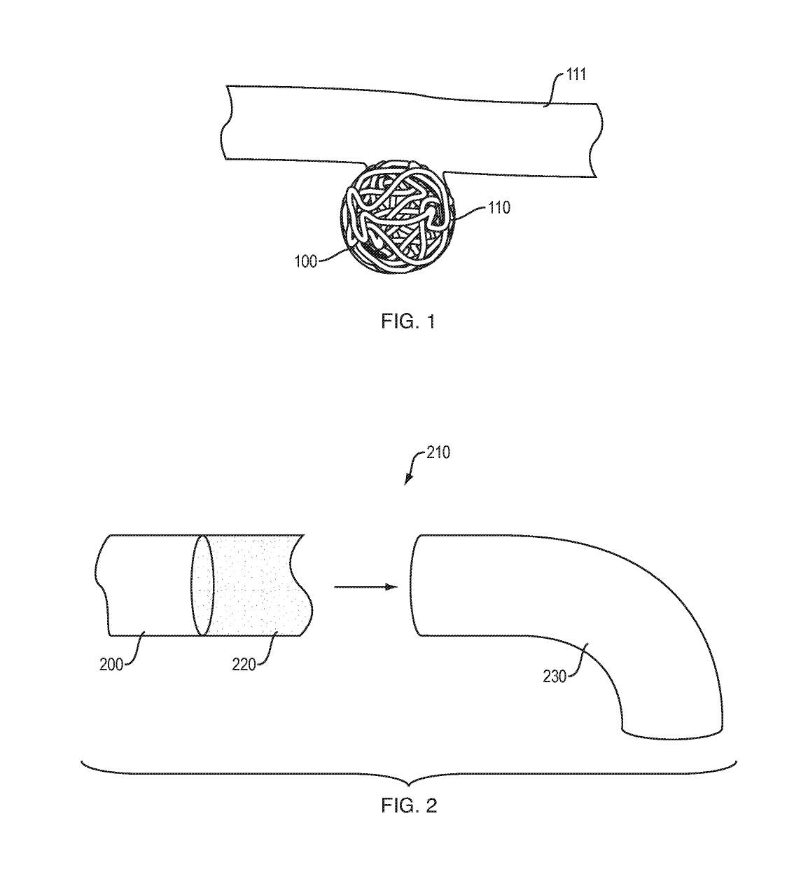

In certain embodiments, the invention is a one-, two- or multi-part foaming system that is deployed into a vessel or cavity. The components of the foaming system react with each other and/or with moisture in the in vivo environment to form a foam. Preferably, the foam forms into a coil or other suitable shaped or unshaped configuration, and thereafter or concurrently expands, and/or solidifies into an embolic structure. Any suitable means are used to deliver the foaming system into the vessel or cavity to be treated. For example, the tip of a delivery catheter may be positioned into the vessel or cavity and the unreacted or partially reacted flowable, formulation material is injected into the vessel or cavity. FIG. 1, for example, illustrates a foam 100 formed into the shape of a coil that has been used to fill an aneurysm 110 that has formed from a blood vessel 111.

In certain embodiments, the in-situ forming foams form an expanding coil during curing and in some embodiments bind together to form an interconnected implant. Other embolization coils known in the art, such as non-expanding coils that do not bind together when formed, are less effective than the methods and systems described herein because the expanding coils provide for improved filling (i.e., occluding) of vessels or other cavities and also reduce the risk of leakage into the vessel or cavity. As used herein, “embolization” is used interchangeably with “occlusion” to mean the partial or complete filling or blocking of a structure. In addition, expanding coils provide for a larger diameter coil to be delivered from a smaller diameter delivery catheter. Small catheters allow for less intrusive percutaneous or minimally-intrusive access within the patient.

In certain embodiments, the delivery catheters used to deliver the polymers of the present invention have a diameter between 4 and 5 Fr (˜1 mm diameter). As the polymer formulation is deployed from the catheter, it either (i) comes into contact with individual formulation components that are delivered separately but concurrently or sequentially, and/or (ii) contacts the aqueous environment in the body (e.g., water and/or blood) to initiate a foaming reaction. In certain other embodiments, the catheter diameter can be much smaller (microcatheters) or much larger (20 Fr or larger). Regardless of the size of the delivery catheter, the polymer formulation is delivered in such a manner so that it preferably forms a coil in certain embodiments, which thereafter foams and expands in diameter and/or length. In such embodiments, the previously cured surface deforms to expose new material to rapidly reform the coil's surface. FIG. 2, for example, illustrates formation of a coil 220 that encounters a moisture containing environment 210 and upon exiting catheter tip 200 forms a foam 230 with a diameter larger than that of the inner lumen of the catheter. The preferred expansion ratio is 1.5 to 5. As is known in the art, the “expansion ratio” is the ratio of volume of foam formed to the volume of formulation used to generate the foam. The preferred kinetics for foam expansion is within two minutes, more preferably within one minute. The preferred kinetics for full curing of the bulk coil is between 1-30 minutes, more preferably between 3-15 minutes. However, the surface reaction to form and hold the coil shape occurs within seconds of contacting water or moisture. The surface of the coil remains tacky such that when one coil contacts another there is some bonding to hold the coils together forming a single implant.

In certain other embodiments, deployment of the formulation is achieved utilizing a simple syringe or power injector attached to a catheter. Alternately, formulation may be supplied in a delivery system that provides a higher-degree of control over the amount of coil deployed. In one such embodiment, the formulation is supplied via a delivery system with a standard connector for catheters. This delivery system consists of a canister that holds the formulation and a plunger to push the formulation out of the canister and into the catheter connector. Control over plunger advancing, and therefore dose, is provided with a screw mechanism, ratchet and lever, electromechanical motor or other system, or other ways known to those skilled in the art.

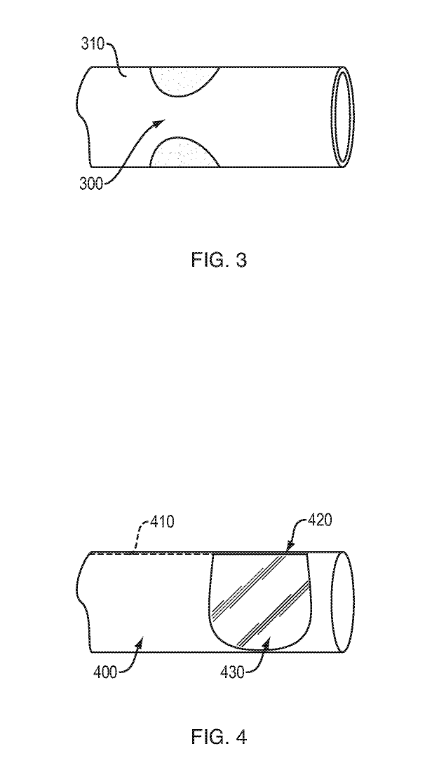

In certain other embodiments, after a dose of formulation is administered, the coil formed may simply break free from the end of the catheter (depending on formulation strength, coil diameter, reaction kinetics, etc.) or it may require an action by the user to detach the coil. Detachment mechanisms may include, for example, the use of a second, coaxial guide catheter that allows the user to shear the coil off at the end. For example, the delivery catheter may deploy the foam through a side port at the distal end, and the coil is sheared off as the catheter is retracted into an outer catheter. Alternately, the delivery catheter may be modified to facilitate detachment of the coil. FIG. 3, for example, illustrates a catheter 310 containing a design feature (e.g, restriction) 300 that provides a weakened area along the coil near the distal end, and/or the catheter may be configured such that heat or other stimuli results in the melting or separation of the polymer coil. This could be provided through a segment of the catheter with reduced diameter 300 or a mesh or partial plug in the lumen of the catheter to reduce the cross-sectional area.

Alternatively, the catheter may be mechanically flexed at a hinge point near the distal tip by advancing a guide wire, retracting the catheter into another catheter or using push/pull wires in the catheter shaft. Alternately, a balloon may be incorporated at the tip or in the lumen of the catheter to reduce the diameter or cutoff flow of the solution—breaking the connection between the deployed formulation from the formulation remaining in the lumen of the catheter. FIG. 4, for example, illustrates a catheter lumen 400 with an inflation lumen 410 that inflates balloon 430 to reduce the diameter of lumen 410. This last approach may also be used to prevent moisture exposure to the unreacted formulation before delivery or in between dosing boluses.

In certain other embodiments, the delivery catheter exposes the unreacted formulation to moisture prior to the formulation exiting the catheter tip. In this fashion a cured surface layer can form or partially form while still in the catheter. This may have several benefits, including enabling the catheter to provide more control over the final shape and size of the coil, providing a more mechanically robust surface layer upon deployment to keep the coil from breaking or bending into collateral vessels and enabling more independent control of chemistry kinetics related to coil formation, expansion / foaming and bulk curing. The exposure to moisture may occur in a very localized segment near the end of the catheter (a few millimeters), throughout the length of the catheter, at discrete and discontinuous segments along the catheter or within the delivery system attached to the catheter. The exposure to moisture may be circumferential, along a discrete arc or more than one discrete and discontinuous arcs. This approach may also be extended to expose a foaming formulation to other components that will induce a reaction (e.g., a catalyst, isocyanate, or polyol). A number of approaches are envisioned to enable this embodiment.

For example, a hydrophilic coating may be employed within the inner lumen of the catheter. Prior to introduction of the formulation, the catheter is flushed with saline, water, water vapor, a hydrophilic material that coats the inner lumen of the catheter, or a solution containing some percentage of water. The water hydrates the hydrophilic coating and when the unreacted formulation passes through that segment a reaction is initiated at the surface. In this embodiment, the surface of the catheter inner lumen may be patterned such that the moisture or hydrophilic material binds to, hydrates and/or adheres to certain portions of the surface. Alternatively, small pores, surface roughness or chambers may be integrated into the inner lumen surface such that water is trapped when flushed through the lumen.

The catheter or a portion thereof may be manufactured from a hydrophilic material or composite that includes a hydrophilic material that hydrates upon flushing or upon contact with blood or other fluid. Hydrophilic materials can be water absorbing cross-linked polymers or composites of such polymers such as: sodium polyacrylate, polyacrylamide copolymer, ethylene maleic anhydride copolymer, cross-linked carboxymethylcellulose, polyvinyl alcohol copolymers, cross-linked polyethylene oxide, and starch/carbonydrate grafted copolymers. As non-limiting examples, small yet potentially sufficient amount of water may be delivered from materials such as ABS, Polysulfone, PAI (polyamide-imide), Radel R, PEEK, Nylon 6, and Nylon 6/6.

The catheter or a portion thereof may be manufactured from a material with pores, holes, slits or other holes that allow moisture from the surrounding bodily fluids to contact the unreacted formulation in the lumen. This may be similar to dialysis tubing which has micropores or microarchitecture to allow water to penetrate through the thickness. For this design, the moisture permeable section may be flexible and pulled into the catheter to facilitate better trackability and placement at the target location. When the catheter is flushed with saline or when the formulation is deployed, the permeable section will be pushed out of the main catheter.

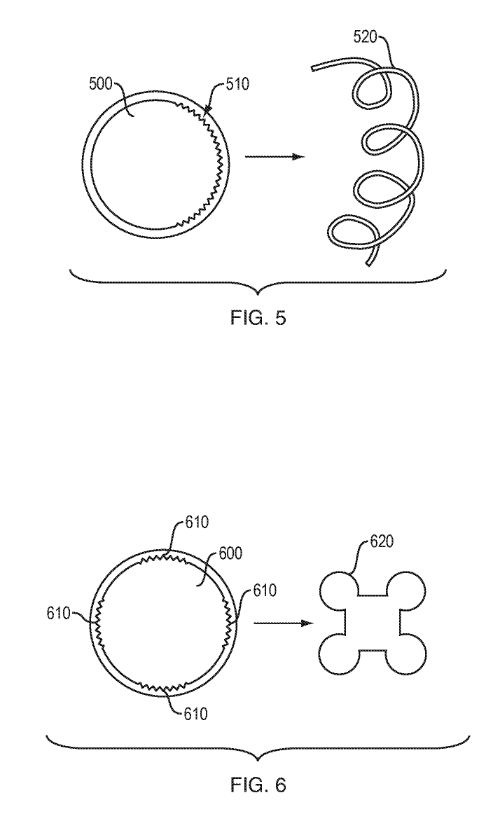

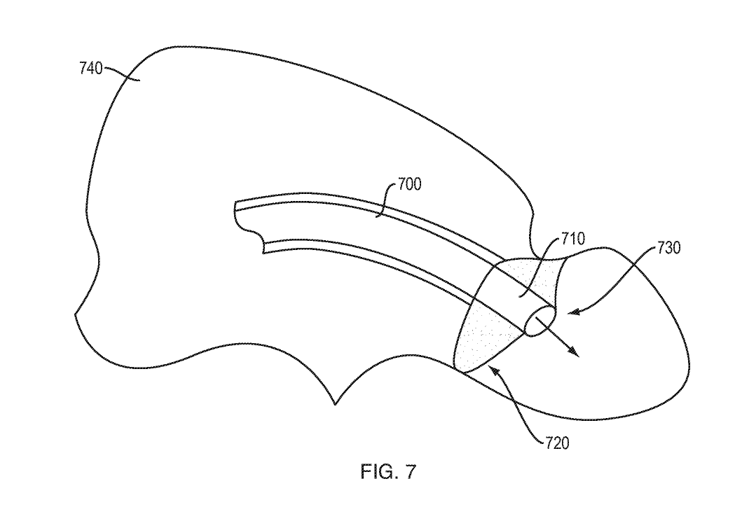

In certain embodiments, these pores, holes or slits that allow moisture from the surrounding bodily fluids to contact the unreacted formulation in the lumen may be non-circumferential around the inner lumen so as to provide preferential surface curing on only a portion(s) of the circumference. FIG. 5, for example, illustrates a moisture permeable region 510 that encompasses approximately one-half of the inner circumference of the lumen 500 such that preferential surface curing releases coil 520. Such preferential surface curing may lead to coiling of the coil upon exit from the catheter tip since a portion will be more cured, and can also provide coils with complex cross-sections because the portions cured within the catheter will be more constrained than those allowed to expand and cure after exit from the catheter tip. FIG. 6, for example, illustrates four separate moisture permeable regions 610 position equidistant around the inner circumference of lumen 600 such that preferential surface curing produces coil 620.

Certain embodiments of the present invention include a catheter tip that is configured to provide complex coil cross-sections, including circular, square, triangular, star shaped, etc.

In certain other embodiments, a plug or coaxial catheter segment may be used in or near the center of the catheter lumen to provide moisture or water on its surface in a manner similar to those described above. In this case, a central core of the coil will cure, providing a mechanical structure around which the remaining formulation can expand and cure. This may also be used in combination with other embodiments described herein to induce curing both in the center of the coil and on the surface.

In certain other embodiments the formulation is provided in an outer tube such that it can be delivered like a more traditional aneurysm coil. The outer tube is dissolvable, soluble or degradable when it contacts water or moisture exposing the formulation in a coil-like structure. For example, materials for this outer tube may include: PEG, PLGA, starch, PPG, a composite or similar materials. The formulation coil will then react with moisture in the environment to expand and cure. The outer tube may also be manufactured from moisture permeable materials, porous materials or perforated materials that then rupture when the formulation begins to react and expand. In this case the rigidity of the unreacted coil (sufficient to enable it to be delivered) may be provided by the outer tube material, the formulation or a combination of both. The viscosity of the formulation may be very high in this case (>5000 cP) or the formulation may be a semi-solid or a solid at room and body temperatures. These formulations may also be reactive to triggers other than moisture, such as pH, temperature, proteins or other factors present in the body. Alternately, the coils may treated or exposed to a trigger that dissolves, melts, degrades or otherwise compromises the outer tube prior to, during or after delivery. For example, an organic solvent, high or low pH fluid, radiofrequency energy, heat, a blade or other mechanical means to score, cut, crush, twist, bend or otherwise rupture the outer tube. Delivery device concepts described earlier in this disclosure may be useful in imparting mechanical means to rupture the outer tube.

In certain other embodiments, a delivery mechanism is attached to the catheter that allows the user to inject a volume of fluid between the unreacted formulation to create discrete lengths of coil. In this design, the delivery system contains the unreacted formulation and an inert, biocompatible substance (e.g., liquid pharmaceutical excipients such as saline, glycerin, lactose, glucose, and gelatin). The two components are in cylinders with plungers and actuation mechanisms that allow the user to dispense each material independently. The exit from each cylinder enters a three-way valve that allows the user to select which material enters the catheter's delivery lumen. In this way, the user can dispense an amount of unreacted formulation that will correspond to a discrete length of coil, then turn the valve and inject an amount of the inert substance into the catheter lumen, then turn the valve again to inject more of the unreacted formulation. This can be repeated and will result in formation of coils of discrete lengths as they exit the tip of the delivery catheter. The delivery system may have markings on it to translate a volume of dispensed foam with a predicted length of coil as it exits the catheter.

In certain other embodiments, the above described embodiments can be modified or used in conjunction with the delivery of therapeutic agents (e.g., chemotherapy agents, proinflammatory, anti-inflammatory, and/or ablative agents such as alcohol). Certain foaming chemistries may also produce therapeutic agents such as alcohol or heat for tissue ablation. The invention may be used in conjunction with available therapeutic agents or may incorporate novel drug delivery approaches such as coaxial fibers that contain therapeutics. The present invention may also be used with liquid, solid, slurry, or semi-solid pharmaceutical preparations that are delivered into vessels or cavities prior to, along with, or after the formulation is delivered. The pharmaceutical preparation and the foam formulation may be delivered through the same or different delivery routes (e.g., catheter and open surgical or catheter and laparoscopic).

### Left Atrial Appendage

The left atrial appendage (LAA) (also known as the left auricular appendix, auricula or left auricle) is a small muscular pouch located high in the left atrium of the heart. The LAA functions as a reservoir for the left atrium and appears to function as a decompression chamber during left ventricular systole and other periods when the left atrial pressure is elevated. Blood clots have a tendency to form in the LAA in patients with atrial fibrillation, mitral valve disease, abnormal contraction of the left atrium and other conditions. These blood clots can dislodge (forming embolic particles), that can travel to tissue and organs (e.g., brain, kidneys, lungs etc.) possibly leading to ischemic damage. In some patients the LAA requires treatment.

In one embodiment, the systems and methods of the present invention relate to the use of one-, two- or multi-part in-situ forming foams for treatment of the left arterial appendage LAA. These foams can be applied to a body cavity and placed into contact with (e.g., deployed into) the LAA for purposes of treating the LAA. When used to treat the LAA, the foams can, among other things, prevent and treat blood clots. More specifically, in certain embodiments, the foams are used to prevent clots from forming in the LAA, stabilizes clots in the LAA, prevent fluid communication between the LAA and the rest of the circulation, and/or prevent changing of the anatomy or function of the LAA.

The components of the foaming system react with each other and/or with moisture in the in vivo environment, and cure, react, expand, and/or solidify into an implant, implant-like structure, or skin. More specifically, the tip of a delivery catheter is positioned into the LAA and the unreacted or partially reacted flowable formulation material is injected into the LAA. The reaction time is preferably short enough to enable the user to complete the procedure in a clinically acceptable time, but long enough to allow adjustments to total foam volume and to allow the foam to interdigitate with surface structures within the LAA. The reaction time is thus preferably between 10-30 minutes. More preferably, the reaction time is between 1-15 minutes. Formulation chemistries that provide for a fast expansion reaction and a slower crosslinking or curing reaction are also preferred. The preferred expansion ratio of the foam is between 1.1× and 100×, and more preferably between 1.5× and 10×. This will provide the user sufficient control over the amount of formulation deployed from the catheter tip and thus the final volume of the foam; excessive expansion ratios are limited in that dispensing small volumes from the catheter tip can be challenging.

In certain other embodiments, the invention is a one- or two-part foaming system that is deployed on the external surface of the heart to constrain the volume of the LAA. This can be accomplished in combination with devices of various configurations. One approach involves the combination of a preformed polymer ring or cuff that serves to constrain the in-situ formulation around the appendage as the forming foam expands. The ring or cuff will be formed from a biocompatible, biostable polymer. In a preferred embodiment, the ring or cuff comprises prepolymer materials that remain substantially unreacted, while prepolymer materials outside of the ring or cuff substantially react to form a form and to compress the ring or cuff to constrain the LAA. The user positions the ring or cuff around the LAA using any suitable mechanism, such as a catheter, endoscope or through open surgery. The formulation is deployed within the circumference of the ring or cuff until compression of the LAA is sufficient to exclude it from blood movement with the left atrium. This can be confirmed during the procedure using standard imaging techniques, such as angiography, ultrasound, or CT scans.

In certain other embodiments, the invention may be used in conjunction with drug delivery, such as procoagulants (thrombin, kaolin, chitosan, fibrin, silica, etc.), proinflammatory agents, or controlled release systems (microspheres, liposomes, monolithic or core-sheath micro and nanofibers, etc.).

In certain other embodiments, fibers or other structures are incorporated into the formulation prior to foam formation, thus yielding a composite structure upon foam formation. Such composite materials can offer mechanical properties that are improved from single material systems.

In certain other embodiments, the invention may utilize a hood on a catheter tip to constrain foam expansion to within the LAA during delivery of the formulation and/or formation of the foam. FIG. 7, for example, illustrates delivery catheter 700 positioned within LAA 730 of the left atrium 740, in which a hood or balloon 720 on catheter tip 710 creates a barrier between LAA 730 and left atrium 740.

In one such embodiment, a polymer film is attached, at one end, concentrically around the tip of a delivery catheter. The other end is attached to the end of a coaxial catheter disposed on the outside of the delivery catheter. As the two catheter tips are brought together the polymer film will flare out and create a hood on the catheter tip. Folds, rods or fibers may be incorporated into the film to control the shape thereof. In particular, stiff polymer fibers may be attached to the film parallel to the catheter axis around the films' circumference with hinge points at the catheter tips and at least one point in between. These will serve to control the shape of the film as it expands when the two catheter tips are brought together.

Constraining foam expansion may also be accomplished with a balloon at the tip of the catheter or a mesh (polymer, nitinol, etc.) used similarly to the film described above. Design of the balloon's fully expanded shape or the mesh's fiber orientation can be used to control the shape upon deployment at the catheter tip. Shape-memory polymers, metals or other materials may also be used to form a mesh plug that expands to exclude the LAA from the left atrium to enable formulation deployment into the LAA.

In certain other embodiments, an Amplatzer or similar plug is deployed into the LAA prior to deploying the formulation into the LAA.

In certain other embodiments, an external approach is used to seal off the LAA prior to foam formation. In such an embodiment, the catheter may be left in the LAA while sealing is undertaken. The formulation is then deployed, and the delivery catheter is removed (which step may include detaching the catheter tip within the LAA).

In certain embodiments, low viscosity, water soluble formulations may be utilized until cross-linked or cured. Such formulations are soluble in water until cross-linked or cured. For example, as the formulation is injected into the LAA, much of it will cross-link or cure and fill the LAA volume whereas any material which exits the LAA will quickly become too dilute to cross-link or cure and will therefore be removed from the body naturally. The intention of delivery will still be to minimize the amount that exits the LAA, so this may be used in conjunction with the other delivery techniques described above.

In certain other embodiments, the previously described formulations that form an implant, implant-like structure, or skin in the LAA can be used to contain the spread of material. In this case, the catheter is placed into the LAA and formulation is deployed. A robust implant, implant-like structure, or skin immediately forms on the surface while new material is incorporated into the bulk. In this way, the formulation will interdigitate with structures within the LAA prior to cross-linking or curing, but the bulk will remain as a single implant. Once the healthcare provider fills the LAA with formulation to the desired amount no further formulation is deployed. The material cures or cross-links filling the LAA space. This may also be accomplished with in-situ coiling formulations, such as those described in the commonly-assigned U.S. patent application entitled “In-situ Forming Foams with Outer Layer,” filed concurrently herewith and naming Freyman et al. as in inventors.

In some embodiments, the foam of the present invention is described to be “lava like” in that it is viscous yet flowable and hardens from its exterior surface towards its interior. The external skin of the foam forms as a fast-forming, robust, balloon-like outer layer that encases the polymer formulation, promotes material cohesion, and resists deformation and movement into collateral vessels or outside the targeted area. As the foam expands this external skin may deform, exposing some of the interior material which then reacts upon contact with the external environment to reform the external skin. The outer layer may be characterized as a “skin” in some embodiments that consists of a thin exterior layer that is more hard or solid, or less flowable, than the material contained by the outer layer. Moreover, the skin may be characterized as being “robust” because it has mechanical properties (e.g., strength, toughness, etc.) that are different, at least for some period of time, to the material contained by the skin. The interior of the material hardens more slowly via the same or a secondary process, as compared to the skin. In some cases where the skin forms rapidly, the material is not cohesive in-situ, resulting in a continuous, packable polymer, which may tend to form as a coil. Through continued extrusion of the material out of a delivery device such as a catheter or microcatheter, the user can create a long coil to partially or completely fill an aneurysm space or other bodily cavity. The space may be filled with an aneurysm coil or other medical implant and an in-situ forming foam or an aneurysm coil that is coated with a material that expands to form a foam coating in-situ. The continuous, long aspect ratio of the coil and cured outer surface prevents the coil from entering the collateral vessels to a significant degree, which could lead to adverse events. These and other factors are important distinctions and advantages of in-situ forming foams over systems and methods that make use of pre-formed foams.

In a preferred embodiment, the foam is formed by a fast cross-linking reaction that can be surface triggered by in-situ water. Multi-functional moisture sensitive silanes are one example of materials susceptible to such reactions especially when formulated with tin, titans or other metal-organic catalysts. One-part cross-linking systems can be created by a two-step process. In the first step, hydroxyl containing siloxanes (either silanols or carbinols) are reacted with an excess of multifunctional silane containing acetoxy, oxime, alkoxy (e.g., methoxy, ethoxy), isopropenoxy, amide, amine, aminoxy, or other functional groups containing silane with the hydrolytically susceptible Si—O—C bond. The resulting prepolymers have multiple groups that are susceptible to hydrolysis. In the second step, such prepolymers are exposed to in-situ water to result in a rapidly cross-linking elastic solid. The reaction proceeds from the outside-in, resulting in a quickly formed outer skin and, in some cases, the formation of the foam into a coil-like configuration. The slower permeation of water or alternative reaction trigger can be used to slowly cure the material inside of the skin. The proteins and pH of the blood can be used to support coil formation by modifying the rate of the skin-forming reaction as well as in coating the formed coil and preventing coil sticking and agglomeration upon self-contact.

Additionally, hydride functional (Si—H) siloxanes or isocyanate functionalized carbinols can be introduced into silanol elastomer formulations to generate gas and produce expanding foamed structures. Expansion of the material can be used to increase the size of the formed coil effectively decreasing coil embolization potential. Expansion of the material can also be critical to increase material size without delivery of more material, in adding porosity and in generating sealing or pressure. Additional formulation ingredients such as surfactants can be used to the impact of generated gas on porosity and expansion.

Alternatively, isocyanate-containing prepolymers are a second example of materials that may be used to generate in-situ forming coils or lava-like foams. Isocyanate groups are relatively unstable when exposed to water and moisture. One-part isocyanate based cross-linking systems can be created by a two-step process. In the first step, polyols, diols, diamines, polyamines, diepoxides, silanols, carbinols or polyepoxides are capped with aliphatic or aromatic diisocyanates such as isophorone diisocyanate (IPDI), hexamethylene diisocyanate (HDI) and methylene diphenyl diisocyanate (MDI). Additionally, multifunctional isocyanates such as HDI biuret, HDI trimer, and polymeric MDI can be combined with diols or diamines. The resulting prepolymers have multiple distant isocyanate groups that are able to react with water and amines found in blood. In the second step, such prepolymers are exposed to in-situ blood resulting in rapid cross-linking and foam formation. The reaction is water-triggered and proceeds from the outside-in, forming a porous outer skin, lava-like shell core structure that assists in coil formation. The expansion of such materials can be important in generating coils of a large diameter while maintaining a small cross-sectional area of the delivery device. Such materials can be used to form stand-alone foaming or gelling coils or combined with each other such that one material is coaxially formed on top the other. For example, a coaxial delivery device can deploy a coil forming formation surrounded by a highly expandable coating formulation. The two formulations may be from different chemistry classes. Alternatively, the two formulations may be selected to be immiscible such that upon delivery the formulations phase separate (e.g., oil miscible and water miscible formulations) to naturally form a coaxial structure. Additionally, the interaction with the catheter wall and/or the density differential of the two fluids can be used to further drive the phase separation. Additionally, two part formulations may be designed such that the two parts are not fully miscible. A surfactant system may be used to formulate the two part formulation into a single stable emulsion. Such an emulsion could be delivered via single chamber delivery device and does not require mixing. The emulsion can be destabilized by shear during delivery or in-situ factors (pH, temperature, ionic strength). Upon such destabilization, the internal phase of the emulsion would spill out and trigger the reaction with the external phase resulting in in-situ foam formation

The solidification of interior portions of foams that form with an exterior skin can be controlled, for example, by altering the permeability of the material to solidification trigger. In the case that the trigger is water, permeability can be controlled by adjusting material hydrophobicity. Additional ingredients can be added to adjust material radiopacity, density, and/or contact angle with blood, tissue, or other biological matrices.

In certain other embodiments, the coils created by the formulation are deployed from catheters, endoscopes, or other minimally-invasive access devices. In addition, the coils created by the formulation are administered from a catheter or syringe during an open surgical procedure.

The present invention offers advantages not previously known in the art. For example, use of the invention will lead to more effective embolics as compared to current treatments because it will result in more effective filling of the vessel or cavity and reduce the risk of leakage into the vessel or cavity or past the embolic. In addition, an expanding coil provides for a larger diameter coil to be delivered from a smaller diameter delivery catheter. Using small catheters allows for less invasive percutaneous or minimally-intrusive access.

In certain other embodiments, there can be a combination of in-situ forming foam with a membrane or other implant which covers the LAA atrial opening. In some embodiments, this membrane or implant comprises fibers or other structures that extend into the in-situ forming foam, thus anchoring it into place.

In certain other embodiments, using open surgery or a minimally-invasive technique (e.g., endoscopy) a bag similar to the shape and size of the LAA is placed over the LAA. In some embodiments, this bag is made from a biocompatible material such as ePTFE, PTFE, polyurethane, etc. While it is held in place over the LAA the formulation is deployed into the bag to collapse the LAA. The formulation chemistry is preferably designed to adhere to the LAA tissue or may be sutured or otherwise attached using techniques known in the art. The foam expansion ratio is preferably between 1.5× and 40×, more preferably 1.5× to 30×. In certain embodiments, the reaction kinetics are such that the foaming will begin within 1 minute of deployment and will be fully cured within 10 minutes. Use of the invention will lead to improved closure of the LAA as compared to current treatments because it will result in more effective seals, will result in a more durable treatment, and will reduce the risk of embolisms arising from the LAA. In addition, a conformal fill of the LAA reduces the risk of movement of the implant, implant-like structure, or skin, and reduces blood leaking into the LAA. In addition, foams may consume less volume prior to deployment and foaming, thus enabling use of lower profile delivery systems and catheters.

### Uterine Artery Embolization

Uterine fibroids are estimated to exist in up to 40% of menstruating women over the age of 50. Uterine fibroids have been treated using PVA particles to embolize the blood supply to the fibroid. During this procedure a small catheter enters the uterine arteries and PVA particles are injected to block the blood supply to the fibroids. After the blood vessels are occluded the distal tissue becomes ischemic and the fibroid tissue necrosis. This tissue is then resorbed by the body during the normal healing process.

In certain embodiments, the present invention comprises the use of a catheter to inject a one-part formulation consisting of an isocyanate-functionalized pre-polymer into the uterine artery(s) or smaller vessels supplying blood to the fibroid. Reaction of the pre-polymer with the blood creates a foam which expands into the vascular network, gels and would lead to occlusion of the vessel(s). This pre-polymer system could additionally contain multiple polymer species, catalysts, surfactants, chain extenders, crosslinkers, pore openers, fillers, plasticizers, and diluents. In the presence of water or blood, the pre-polymer phase reacts to form a foam. The viscosity of this pre-polymer is preferably less than 5000 cP and more preferably less than 2000 cP. This approach would lead to more complete occlusion of the vessel compared to the current PVA particle approach because the macro-scale foam would have less tendency to migrate than 500 um PVA particles. Additionally, a balloon could be inflated at the distal end of the catheter to prevent retrograde flow of the pre-polymer or foam and to ensure that foam transported only towards the fibroid. Foams for this application would be absorbable or non-absorbable and biocompatible. Foams could also be dissolvable; to do so, specific chemical links would be incorporated into the chemistry. After 20 minutes a second agent could be added to dissolve those links enabling the foam to be dissolved and aspirated away.

### Arteriovenous Malformations

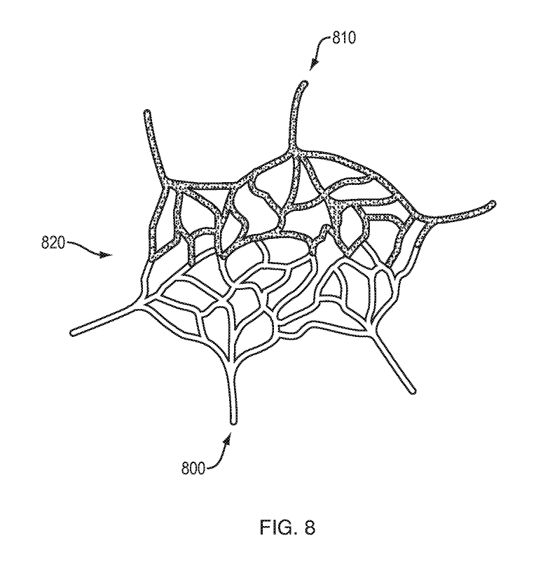

An arteriovenous malformation (AVM) is an abnormal condition between the arteries 800 and veins 810 of a capillary network 820, typically occurring in the central nervous system, as shown in FIG. 8. AVM can be treated by embolization with Onyx or using coils to embolize. As described above, our one-part polymer system could also be used to embolize the AVM. AVMs, both low and high flow, in all parts of the body could be treated, including cerebral, femoral, pelvic AVMs.

### Male and Female Sterilization

Obstruction of the fallopian tubes in females or vas deferens in males will lead to sterilization. It is desirable to be able to reverse this process by re-opening these lumens at some later time. Although foam delivery through a catheter, syringe, or other suitable delivery means is possible, in the preferred embodiment a device consists of a semi-porous balloon filled with a pre-polymer or one part of a two part foam. One end of the balloon as a one-way (e.g., duckbill) valve through which water or the second part of a two part foam can be infused. Once this second component is introduced the materials will foam and expand. This action will expand the balloon to occlude the fallopian tube or vas deferens into which it was inserted. The balloon can be non-compliant (i.e., will be sized by the operator to fit the target lumen), semi-compliant or compliant. In the latter two cases, the amount of foam components introduced to the balloon will impact the final diameter and/or outwards force exerted on the lumen wall. The balloon wall will be porous to allow some of the foam to escape and prevent device movement and create a better seal around the implant. These pores will be between 0.1 microns to 1 mm in diameter. More preferably the diameter will be between 50 microns to 1 mm. Also, these pores may take the form of longitudinal or transverse slits in the balloon surface. Other pore shapes and distribution geometries are also contemplated. Pore distribution need not be uniform along the length of the balloon. For example, the ends or last 5 mm of length on each end may be non-porous. This will prevent foam expansion beyond the balloon length. In addition, the balloon may be non-porous. In this case the balloon may have a texture such that when fully inflated the texture increases the traction on the lumen wall. This prevents migration and improves the seal. The balloon may also have a non-uniform diameter along its length for this same purpose. For example, the balloon may be hourglass shaped, tapered or corrugated. For use in females the balloon diameter will be in a range of 0.4 to 2.5 cm. More preferably in a diameter of 0.5 to 2 cm. For use in males the balloon diameter will be in a range of 0.1 to 10 mm. More preferably in a diameter of 2 to 5 mm.

### Diverticular Bleeding

In this condition the patient has bleeding in the distal portion of the digestive tract from diverticula. They are often numerous and it is difficult to identify the source of bleeding. A hemostatic foam will be deployed into the lumen. The delivery system will be inserted into the anus and have an inflatable balloon on the distal portion. A catheter lumen will extend beyond the balloon. When the balloon is inflated it will direct foam expansion (after deployment through the catheter lumen) into the digestive tract; preventing retrograde movement. The result will keep expanding foam within the targeted portion of the digestive tract. The foam can be removed minimally invasively using skills known in the art. Alternatively, the formulation can be designed such that after exposure to another agent it degrades and can be passed. In yet another embodiment, the foam can be designed to collapse as the windows between cells rupture due to mechanical forces from bowel motion.

### Ear Canal Indications

Foams may be used to obstruct or seal the ear canal for a variety of indications. In one embodiment, a formulation can be used to make an ear plug that foams up and stays in place. The formulation can come in two parts having a putty-like consistency, and a user will knead the two part putty together to mix them. The formulation will generate a low-expansion foam, which can be formed into a shape that fits easily into the ear canal. After the putty is inserted into the ear, it expands to form a seal. This embodiment may be particularly useful for young children, or for nose-bleed applications. The formulation may also include a drug or drugs that are useful for various indications, such as treatment of ear infections.

Other commercial applications for in-situ forming foams include treatment of: bleeding after dilation and curettage (D&C); post-hysterectomy bleeding; uterine AV fistulas; liver or lung tumor resection; HHT fistula; GI bleeding.

Some of the advantages that this invention provides over the current state of the art include the following: ability to deliver into a closed cavity (intravascularly); ability to reach inaccessible sites; ability to expand into empty space or space filled with blood; ability to displace blood from a space; and ability to fill a cavity or a defect.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

We claim:

1. A method of at least partially occluding a cavity within a patient, comprising the steps of:
providing a fluid prepolymer material,
delivering said fluid prepolymer material into said cavity, and
forming a foam within said cavity from said fluid prepolymer material.

2. The method of claim 1, wherein said step of delivering said fluid prepolymer material is conducted with a delivery device that comprises a catheter.

3. The method of claim 1, wherein said step of delivering said fluid prepolymer material is conducted with a delivery device that comprises an endoscope.

4. The method of claim 1, wherein said foam is formed by the reaction of said fluid prepolymer material in the presence of a water-containing environment to generate a gas.

5. The method of claim 4, wherein said foam is an expanding foam formed by the reaction of said fluid prepolymer material in the presence of a water-containing environment to generate a gas.

6. The method of claim 4, wherein said water-containing environment comprises blood.

7. The method of claim 1, wherein said cavity is a blood vessel.

8. The method of claim 1, wherein said cavity is a left arterial appendage.

9. The method of claim 2, wherein said fluid prepolymer material is delivered into a space between the exterior surface of said catheter and said cavity.

10. The method of claim 7, wherein said foam at least partially occludes a uterine artery.

11. The method of claim 7, wherein said foam at least partially occludes an arteriovenous malformation.

12. The method of claim 1, wherein said foam at least partially occludes a fallopian tube.

13. The method of claim 1, wherein said foam at least partially occludes the vas deferens.

14. The method of claim 7, wherein said foam is used to treat post-hysterectomy bleeding.

15. The method of claim 7, wherein said foam is used to treat a uterine AV fistula.

16. The method of claim 7, wherein said foam is used to treat diverticular bleeding.

17. The method of claim 7, wherein said foam is used to treat gastrointestinal bleeding.

18. The method of claim 7, wherein said foam is delivered following a liver resection.

19. The method of claim 7, wherein said foam is delivered following a lung resection.

20. A method of at least partially occluding a cavity within a patient, comprising the steps of:
providing a fluid prepolymer material,
delivering said fluid prepolymer material into said cavity, and
forming a foam within said cavity from said fluid prepolymer material;
wherein said foam is characterized with an expansion ratio within a range of 1.5-5.0, and said foam is fully cured within 10 minutes after said step of delivering said fluid prepolymer material into said cavity.

21. The method of claim 20, wherein said form is fully cured within 1 minute.
